(19) United States
(12) Patent Application Publication
(10) Pub. No.: US 2014/0044731 A1
(43) Pub. Date: Feb. 13, 2014

(54) METHODS OF TREATING AGE-RELATED MACULAR DEGENERATION

(71) Applicants: Robert Nussenblatt, Bethesda, MD (US); Baoying Liu, Gaithersburg, MD (US); Lai Wei, Rockville, MD (US)

(72) Inventors: Robert Nussenblatt, Bethesda, MD (US); Baoying Liu, Gaithersburg, MD (US); Lai Wei, Rockville, MD (US)

(21) Appl. No.: 13/977,946

(22) PCT Filed: Jan. 3, 2012
(86) PCT No.: PCT/US12/20050

§ 371 Date: Oct. 17, 2013

(30) Foreign Application Priority Data
Jan. 4, 2011 (US) 61429580

(51) Int. Cl. A61K 39/395; G01N 33/68

(52) U.S. Cl. CPC A61K 39/3955; G01N 33/6893
USPC 424/158.1; 435/7.92

(57) ABSTRACT

The present invention is directed to methods of treating and diagnosing age-related macular degeneration.

### CROSS-REFERENCE TO RELATED APPLICATIONS

This application claims the benefit of U.S. Provisional Application No. 61/429,580, filed Jan. 4, 2011, the entirety of which is incorporated herein.

### TECHNICAL FIELD

The present invention is directed to methods of treating age-related macular degeneration.

### BACKGROUND

Age-related macular degeneration (AMD) is the leading cause of blindness in the United States and Europe and is the most common cause of irreversible blindness in the older population worldwide. As such, methods for detection and treatment of AMD are needed.

### SUMMARY

The present invention is directed to methods of treating age-related macular degeneration in a patient comprising administering to the patient a therapeutically effective amount of an anti-IL-22 antagonist. The invention is also directed to assays for use in a patient having or suspected of having macular degeneration, comprising: determining the concentration of IL-22 in a serum sample from said patient, wherein an elevation in the level of IL-22 in said serum sample, relative to the concentration of IL-22 in the serum of patients not having macular degeneration, is indicative of the presence or severity of age-related macular degeneration in said patient.

### BRIEF DESCRIPTION OF THE DRAWINGS

FIG. 1 suggests that C5a promotes the expression of IL-22 and IL-17 from T cells. (A) PBMCs from 2 control donors and 2 AMD patients were cultured with or without C5a overnight. CD3+CD4+ T cells were sorted and RNAs were purified for microarray analysis. (B) IL-22 and IL-17 in 3-day culture supernatants of PBMCs from 14 AMD patients and 14 controls. (C) C5a induced IL-22/IL-17 expression in both controls and AMD patients were subgrouped based on CFH genotypes. (D) Intracytoplasmic staining of IL-22 and IL-17 from both controls and AMD patients after 5 days of culture with or without C5a and C5aR antagonist.

FIG. 2 suggests that IL-1β and IL-6 secreting monocytes are important for C5a induced IL-22 and IL-17 expression form T cells. (A) CD3⁺CD4⁺ T cells and CD3⁻CD14⁺ monocytes were sorted and cultured with or without C5a for 4 days. Cell supernatants were assessed for IL-22 and IL-17 expression. (B) CD3⁺CD4⁺CD45RA⁺ and CD3⁺CD4⁺CD45RA⁻ T cells and CD3⁻ CD14⁺ monocytes were sorted and cultured with or without C5a for 4 days. IL-22 and IL-17 levels were measured from supernatants. (C) IL-22 and IL-17 in 4-day culture supernatants of PBMCs with the presence or absence of C5a, C5aR antagonist and anti-B7.1 and anti-B7.2 antibodies. (D) IL-22 and IL-17 in 4-day culture supernatants of PBMCs with the presence or absence of C5a, C5aR antagonist and anti-IL-1β and anti-IL-6 neutralization antibodies. (E) CD3⁺CD4⁺ T cells were sorted and stimulated with anti-CD3, anti-CD28 with or without IL-1β and IL-6 for 24 hours. RNAs were purified for RT-PCR analysis of BATF.

FIG. 3 suggests that C5a protects T cells from undergoing apoptosis. (A) Top 4 affected gene ontology enrichment analysis by C5a in the microarray analysis. (B) Annexin V expression on T cells cultured with or without C5a and C5aR antagonist. (C) PBMCs were treated with or without C5a for 3 days. T cells were sorted and processed for western blot analysis for indicated antibodies. Similar results were seen in another independent assay.

FIG. 4 suggests that IL-22 and IL-17 present a higher expression in the serum of AMD patients. Serum from 29 controls and 25 AMD patients were assayed for IL-22. Thirty (30) controls and 23 AMD patients was assayed for IL-17 expression. IL-22/IL-17 expression in both controls and AMD patients were subgrouped based on the subjects' CFH genotypes.

FIG. 5 suggests that C5a activates B7 expression on monocytes. PBMCs were cultured with or without C5a for 1 day. CD3⁻CD14⁺ monocytes were gated for indicated cell markers' expression. Similar results were seen in another independent assay.

FIG. 6 suggests that C5a stimulates monocytes to secrete IL-1β and IL-6. (A) PBMCs were cultured with or without C5a and C5aR antagonist for 3 days. Cell supernatants were assayed for IL-1β, IL-6 and TNFα expression. (B) Monocytes and T cells were sorted and cultured with or without C5a for 3 day. Cell supernatants were assayed for IL-1β and IL-6 expression.

FIG. 7 depicts the results of vitreous from one age matched control and one AMD patient were assayed for IL-22 expression.

FIG. 8 Human adult retinal pigmented epithelium cells were treated with or without IL-22 (50 ng/ml) for 3 days. MTT assay was performed to detect cell survival and proliferation. The experiment was repeated 3 times and representative results are depicted in FIG. 8.

FIG. 9 depicts that IL-22 decreased total tissue resistance and induced apoptosis of primary human RPE cells. Monolayers of RPE cells grown on inserts were treated with recombinant human IL-22 (50 ng/ml). Resistance measurements and an apoptosis assay were performed after 72 h of incubation with IL-22. (A) Summary bar graph of TER changes after addition of IL-22 to human fetal RPE monolayers. Compared with matched controls, TER significantly (60%) decreased in IL-22-treated monolayers of RPE (n=3, p<0.01). (B) A histogram comparing annexin V staining for human primary RPE cells with and without IL-22 treatment. The gray line is for the control group while the bolded dark line is for IL-22-treated RPE cells. There is a right shift of annexin V staining for IL-22-treated RPE cells when compared with the nontreated control group, indicating increased apoptosis induced by IL-22 treatment. (C) Monolayers of RPE cells grown on inserts were treated with or without recombinant human IL-22 (50 ng/ml) for 48 h and cells were lysed and subjected to Western blot analysis for phosphorylated Bad. Although the β-actin levels were comparable comparing IL-22-treated and untreated groups, the phosphorylated Bad was decreased after IL-22 treatment.

### DETAILED DESCRIPTION OF ILLUSTRATIVE EMBODIMENTS

It has now been discovered that C5a promoted interleukin-22 (IL-22) and interleukin-17 (IL-17) expression from human CD4⁺ T cells of AMD patients and normals is accompanied by a higher expression of transcription factor BATF. Also, significantly increased levels of IL-22 and IL-17 were identified in the serum of AMD patients and increased IL-22 expression was identified in the vitreous of an AMD patient, as compared to patients not having AMD. It has also been found that IL-22 directly decreased retinal pigment epithelial (RPE) cell viability.

The present invention is directed to methods of treating age-related macular degeneration in a patient. Methods of the invention can be used for the treatment of wet age-related macular degeneration and dry age-related macular degeneration. The methods of the invention comprise administering to the patient a therapeutically effective amount of an anti-IL-22 antagonist.

Anti-IL-22 antagonists, for example, anti-IL-22 antibodies, are known in the art, albeit not for the treatment of age-related macular degeneration. Such antagonists are described in, for example, U.S. Published App. No. 20090093057, U.S. Published App. No. 20050042220, and U.S. Published App. No. 20090220519, the entireties of which are incorporated herein in their entireties.

The term “antagonist” is used in the broadest sense, and includes any molecule that partially or fully blocks, inhibits, or neutralizes a biological activity of IL-22. Suitable antagonist molecules specifically include antagonist antibodies or antibody fragments, fragments or amino acid sequence variants of IL-22, peptides, antisense oligonucleotides, small organic molecules, etc. Methods for identifying antagonists of IL-22 may comprise contacting IL-22 with a candidate antagonist molecule and measuring a detectable change in one or more biological activities normally associated with IL-22.

The invention is also directed to assays for use in a patient having or suspected of having macular degeneration. These methods comprise determining the concentration of IL-22 in a serum sample from said patient, wherein an elevation in the level of IL-22 in said serum sample, relative to the concentration of IL-22 in the serum of patients not having macular degeneration, is indicative of the presence or severity of age-related macular degeneration in said patient.

The term “therapeutically effective amount” refers to the amount of the anti-IL-22 antagonist effective to achieve the desired therapeutic effect.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

### Experimental Section

### Methods

Cell sorting. PBMCs were obtained from the peripheral blood of AMD patients and healthy subjects in compliance with institutional review board (IRB) protocols after informed consent. AMD subjects were diagnosed with wet AMD without accompanied systemic autoimmune diseases or other immune-related diseases by experienced clinicians. CD4⁺ T cells and monocytes were subsequently stained with the following antibodies: CD3⁺CD4⁺, CD3⁺CD4⁺CD45RA⁺, CD3⁺CD4⁺CD45RA⁻, CD3⁻CD14⁺ and were sorted on a FACS Aria (BD Biosciences).

Microarray. Approximately 10 μg of RNA was labeled and hybridized to Genechip human genome U133 plus 2.0 array (Affymetrix) according to the manufacturer's protocols. Expression values were determined with GeneChip Operating Software (GCOS) v1.1.1 software. All data analysis was performed with GeneSpring software GX 7.3.1 (Agilent Technologies).

Cell Culture and Flow Cytometry.

PBMC Cells were treated with or without C5a (50 ng/ml) and a C5aR antagonist (2.5 ug/ml). Anti-B7.1 and B7.2 antibodies (10 μg/ml of each) or anti-IL-1β (10 μg/ml) and anti-IL-6 (10 μg/ml) neutralization antibodies were added into the cell culture in indicated experiments. Supernatants were collected and tested by ELISA for IL-22 and IL-17, or sent for multiplex cytokine analysis (Aushon Biosystems). Intracellular staining were performed after 5 days of C5a culture and stained with FITC-CD4, PE-IL-22 or PE-IL-17A and APC-CD3. Cells were stimulated with PMA (10 ng/ml), ionomycin (0.5 μg/ml) and Golgistop for 4 hours at 37° C. before intracellular staining. C5R staining was performed using 3-step procedure. 10 μg/ml mouse anti-human C5aR (clone: D53-1473) was used for staining C5aR on the T cells and then biotin-rat anti-mouse IgG1 (10 μg/ml) and streptavidin-APC (4 μg/ml) were sequentially engaged to detect C5aR expression. Human adult retinal pigment epithelium cells were kindly provided by Drs. Hooks and Nagineni at Laboratory of Immunology, National Eye Institute. These cells were derived from an 89 year old donor eye from the New England Eye bank. Cells were grown in MEM supplemented with 10% FBS and non-essential amino acids.

Real-Time PCR.

One million T cells were stimulated with anti-CD3 (0.1 μg/ml) and anti-CD28 (1 μg/ml) with or without IL-1β (10 ng/ml) and IL-6 (25 ng/ml) overnight. Cells were then lysed in 250 μl lysis/binding buffer. RNA was isolated using mirVana™ miRNA isolation kit (Ambion). Total RNA was converted to cDNA using Tagman reverse transcription reagents (Applied Biosystems). Quantitative PCR was performed using a 7500 Fast Real-time PCR system (Applied Biosystems). BATF and 18S ribosomal RNA primers and probes were obtained from Applied Biosystems and used accordingly to standard methodologies.

Apoptosis Assay.

Apoptotic cells were detected by staining cells with the annexin-V-FITC according to the manufacturer's instructions (BD Biosciences). Phopho-Bad expression was detected by western blot using anti-phospho-Bad antibody (Cell Signaling Technology).

SNP Genotyping.

Genomic DNA was extracted from the peripheral blood of each individual using Promega Wizard Genomic DNA Purification kit. The samples were analyzed by TaqMan genotyping assay using the Real-time PCR system 7500 (Applied Biosystems, Foster City, Calif., USA). The primers and probes for C2/CFB rs9332739 and C3 rs2230199 were from the inventory SNP assay while CFH rs1061170 were custom-designed from Applied Biosystems. Genotypes were determined based on the fluorescence intensities of FAM and VIC. The call rates of 3 assays were >98.5% and the call accuracies (consistency of duplicate wells of selected samples) were 100%.

Statistical Analysis.

Non-parametric methods were used since IL17 and IL22 do not follow a parametric distribution. For the association study between IL-22/IL-17 and some characteristics of patients (CFH, C3 genotypes, gender, co-morbidities of diabetes, hypertension and hypercholesterolemia), Wilcoxon's nonparametric two-sample rank sum test was used. Age was analyzed using Pearson correlation.

The demographic, clinical information for both controls and AMD patients is listed in Table 1 and Table 2. All the subjects in this study are Caucasians. There are 45 controls and the age range was from 59 to 87. Fifty-three percent (53%) are females and 47% are males. There are 40 AMD patients in this study and the age range was from 57 to 97. Fifty percent (50%) are females and 50% are males.

C5a Promotes the Expression of IL-22 and IL-17 from T Cells.

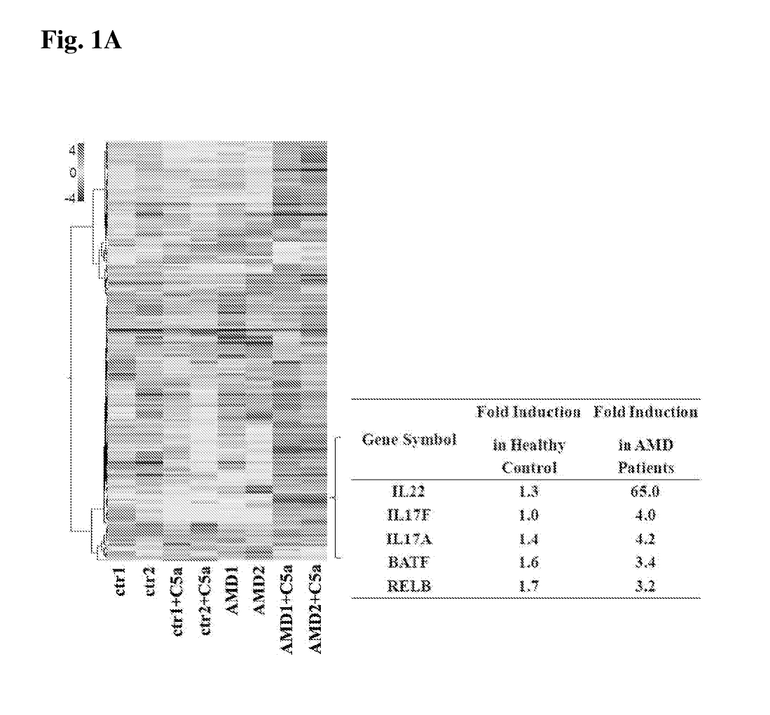

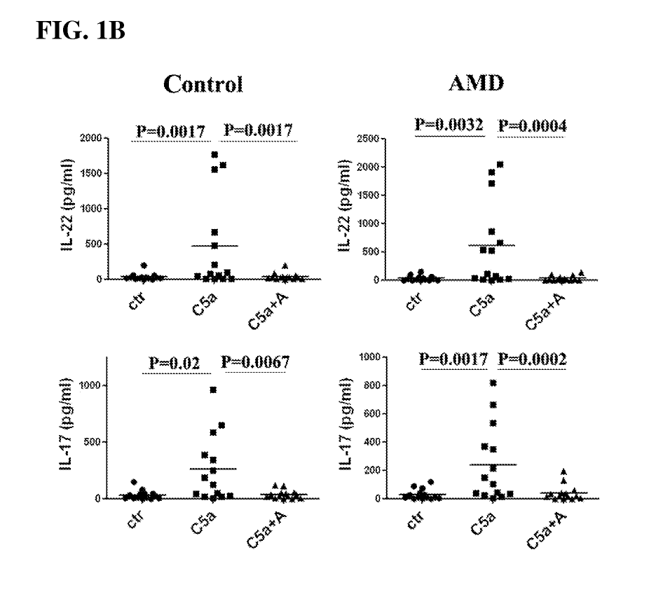

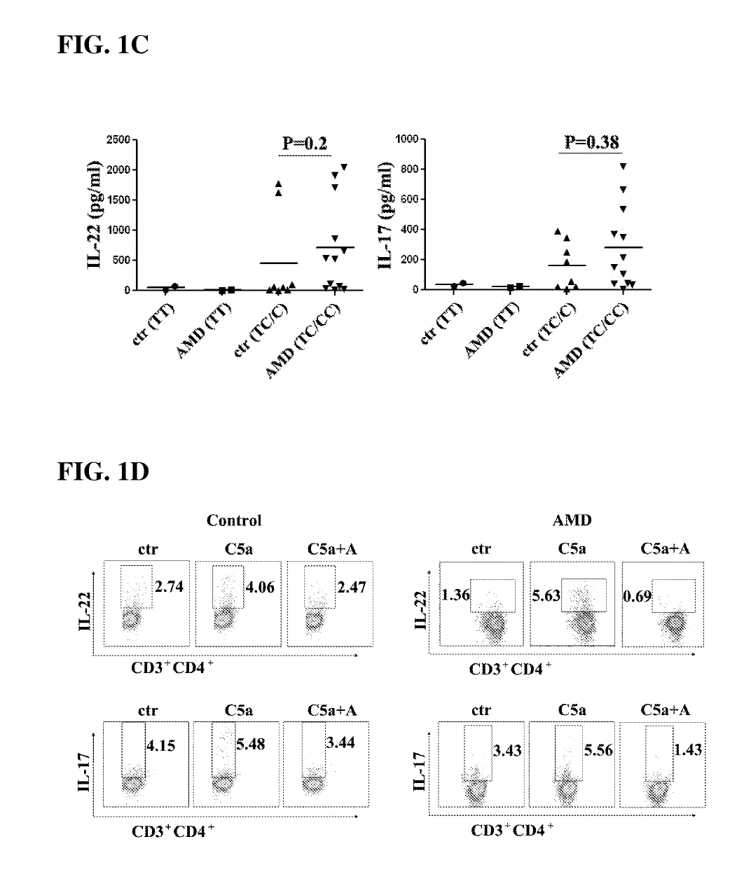

To study the role of C5a on CD4⁺ T cells in AMD patients, genome-wide expression profiling was performed using the Affymetrix GeneChip U133 plus 2.0 arrays. Parametric one-way ANOVA (p<0.05) identified 168 probe sets (representing 132 unique genes) whose expression was different by at least two fold between cells from any two of four groups (64 and 77 year old female controls, C5a treated controls, 78 and 80 year old female AMD, C5a treated AMD) (FIG. 1A, Table S). Although the majority of these genes were regulated by C5a in a similar manner in healthy controls, and AMD patients, a set of genes were differentially induced by C5a between the two groups of subjects. Surprisingly, C5a did not change the expression of IL22 in cells from healthy controls. However, a 65 fold induction of IL22 by C5a was found in the cells from AMD patients, and was the most differentially changed gene by C5a between healthy controls and AMD patients. In addition to IL22, IL17A, IL17F, as well as BATF were also highly induced by C5a only in AMD patients but not in healthy controls (FIG. 1A, Table S).

TABLE S

| Gene | Fold Change | Fold Change | | | | | | | | |
|---|---|---|---|---|---|---|---|---|---|---|
| Symbol | Ctr vs C5a | AMD vs C5a | Ctr1 | Ctr2 | Ctr/C5a1 | Ctr/C5a2 | AMD1 | AMD2 | AMD/C5a1 | AMD/C5a2 |
| IL22 | 1.3 | 65.0 | 4.9 | 5.9 | 7.5 | 6.8 | 6.7 | 6.6 | 353.3 | 508.2 |
| IL17F | 1.0 | 4.0 | 16.7 | 14.7 | 17.6 | 12.8 | 17.4 | 14.2 | 54.5 | 72.3 |
| IL17A | 1.4 | 4.2 | 6.9 | 6.8 | 11.9 | 7.6 | 7.2 | 6.5 | 27.7 | 29.7 |
| SLC1A4 | 1.5 | 3.9 | 151.4 | 89.3 | 192.6 | 178.6 | 83.0 | 61.7 | 274.8 | 293.0 |
| CCL20 | 3.4 | 8.4 | 160.7 | 95.7 | 480.9 | 397.7 | 67.5 | 42.2 | 289.5 | 628.1 |
| ICAM1 | 1.2 | 3.0 | 39.7 | 28.4 | 52.1 | 32.6 | 29.0 | 23.8 | 92.3 | 64.6 |
| PRF1 | 0.8 | 2.0 | 161.4 | 278.5 | 128.2 | 244.4 | 353.5 | 362.5 | 762.7 | 674.8 |
| MAP3K8 | 1.1 | 2.6 | 32.5 | 29.7 | 37.4 | 33.3 | 30.3 | 32.8 | 79.8 | 81.9 |
| SLC27A2 | 1.1 | 2.4 | 16.6 | 21.7 | 19.8 | 21.6 | 19.4 | 15.0 | 37.1 | 46.6 |
| PFKFB3 | 2.0 | 4.0 | 1218.4 | 605.3 | 1756.8 | 1816.9 | 456.3 | 544.7 | 1695.4 | 2351.0 |
| BATF | 1.6 | 3.4 | 470.4 | 433.6 | 889.8 | 595.8 | 459.0 | 553.4 | 2035.7 | 1394.8 |
| AKR1CL2 | 0.5 | 0.9 | 51.1 | 43.9 | 22.8 | 21.1 | 41.7 | 45.6 | 38.9 | 38.7 |
| PMAIP1 | 1.3 | 2.4 | 23.7 | 23.0 | 36.8 | 23.4 | 32.8 | 30.1 | 56.9 | 95.0 |
| CD83 | 1.7 | 3.1 | 64.9 | 62.8 | 105.4 | 109.0 | 69.8 | 77.3 | 205.7 | 254.3 |
| NAMPT | 1.7 | 3.2 | 156.8 | 116.2 | 272.4 | 195.9 | 117.1 | 118.0 | 367.0 | 381.8 |
| RASGRP3 | 1.3 | 2.3 | 40.6 | 37.8 | 54.2 | 44.4 | 36.4 | 39.1 | 93.8 | 82.1 |
| ZNRF1 | 3.1 | 5.7 | 126.9 | 49.1 | 373.7 | 172.3 | 60.5 | 81.6 | 481.9 | 334.4 |
| PLEKHG2 | 1.4 | 2.7 | 117.9 | 76.2 | 162.0 | 119.1 | 85.2 | 76.9 | 257.7 | 174.9 |
| PPP1R3B | 1.9 | 3.6 | 202.9 | 141.4 | 390.8 | 278.0 | 144.0 | 153.1 | 612.2 | 445.1 |
| SGK1 | 2.3 | 4.1 | 196.1 | 123.9 | 444.7 | 281.5 | 220.6 | 219.0 | 1131.6 | 686.9 |
| RELB | 1.7 | 3.2 | 89.2 | 113.6 | 189.2 | 164.1 | 104.5 | 138.2 | 401.7 | 363.1 |
| HLF | 0.9 | 1.6 | 46.5 | 41.5 | 43.4 | 35.6 | 18.4 | 22.4 | 35.1 | 31.2 |
| NFKBIA | 1.8 | 3.1 | 851.6 | 894.2 | 1665.8 | 1390.4 | 951.5 | 1154.5 | 3252.8 | 3188.5 |
| FAM46C | 1.4 | 2.5 | 923.5 | 700.7 | 1198.7 | 1139.5 | 682.6 | 371.1 | 1333.9 | 1305.0 |
| CD200 | 1.5 | 2.5 | 99.6 | 134.3 | 151.3 | 188.5 | 74.2 | 85.8 | 185.7 | 213.5 |

To determine whether the changes in mRNA translated into differences in protein production, ELISA and intracellular staining was used to validate the microarray data. PBMCs from AMD patients and controls were treated with or without C5a and a C5aR antagonist (Jerini Ophthalmic, Inc) for 3 days. Cell supernatants from 14 controls and 14 AMD patients were used for ELISA analysis and are presented side by side in FIG. 1B. In agreement with the microarray results, the addition of C5a greatly increased the expression of IL-22 and IL-17A in PBMC cells from AMD patients. Although gene expression profiles indicated that the promotion of Th17 cytokines and regulators by short C5a treatment (24 h) was selective in cells from AMD patients (FIG. 1A), similar effects were also observed in controls. Blocking C5aR reversed this effect (FIG. 1B). The C5a induced IL-22/IL-17 expression was then subgrouped in both controls and AMD patients based on their CFH SNP information (rs1061170). As shown in FIG. 1C, there was no significant difference on cytokine expression between controls and AMD patients. However, C5a high response individuals all have the risk CFH allele genotype (heterozygous/homozygous, TC/CC) in both control and patient groups. Intracellular staining data further confirmed that C5a induced IL-22 and IL-17A secretion from cultured CD3⁺CD4⁺ T cells after PBMCs were treated for 5 days (FIG. 1D).

Monocytes are Important for C5a Induced IL-22 and IL-17 Expression Form T Cells.

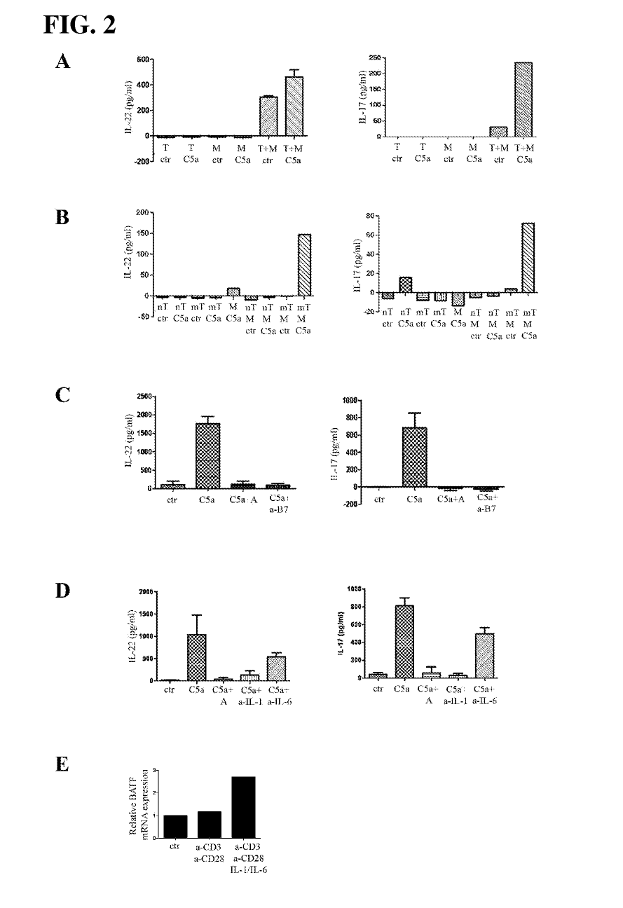

CD14⁺ monocytes and CD3⁺CD4⁺ T cells were cultured separately or together, with or without C5a (50 ng/ml) for 72 hours. Protein levels of IL-22 and IL-17A in the culture supernatants were detected by ELISA. As shown in FIG. 2A, IL-22 and IL-17 were barely detected in cultures with monocytes or CD4⁺ T cells alone. Interestingly, C5a induced expression of both cytokines only in co-cultures of CD4⁺ T cells and monocytes, suggesting that monocytes are necessary for C5a to promote IL-22 and IL-17 expression. Further experiments showed that only memory CD4⁺ T cells, when co-cultured with monocytes, could produce Th17 cytokines (FIG. 2B).

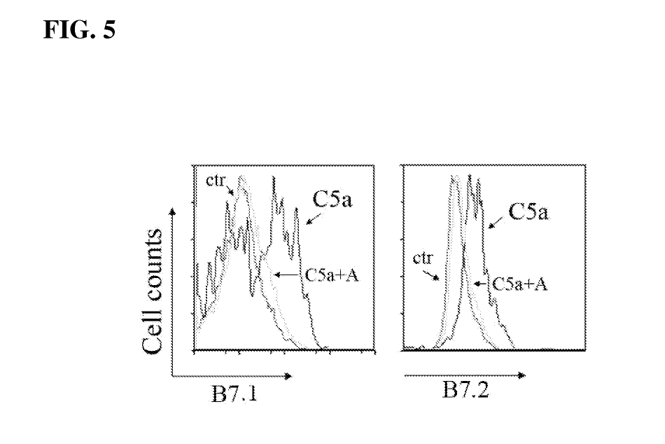

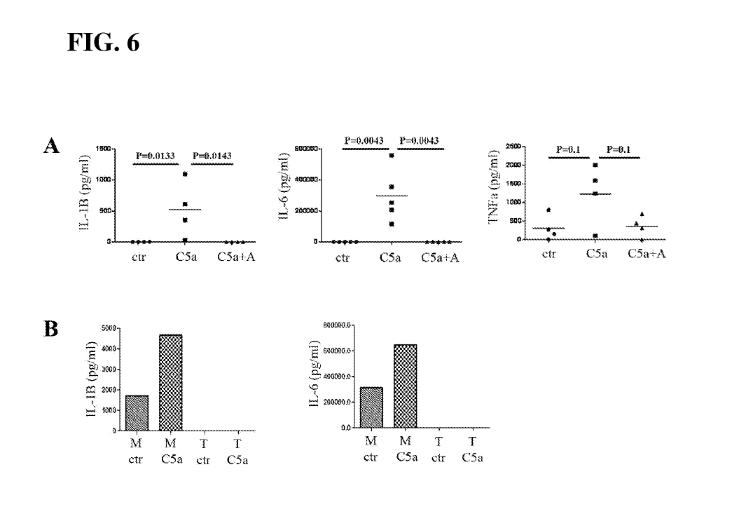

The effects of monocytes on T cells could be due to either direct interaction between B7.1/B7.2 on monocytes and CD28 on T cells, or indirect effects such as the production of cytokines. C5a treatment promoted both B7.1 and B7.2 expression on monocytes (FIG. 5). When a blocking antibody that interrupts the B7-CD28 interaction was added to the culture, the induction of both IL-22 and IL-17 by C5a was diminished, to a similar extent as the effect seen with the C5aR antagonist (FIG. 2C). Previous studies have shown that IL-1β and IL-6 are drivers of Th17 cell polarization 9 (Volpe E, et al. (2008) A critical function for transforming growth factor-beta, interleukin 23 and proinflammatory cytokines in driving and modulating human T(H)-17 responses. Nature immunology 9(6):650-657; Acosta-Rodriguez E V, Napolitani G, Lanzavecchia A, & Sallusto F (2007) Interleukins 1beta and 6 but not transforming growth factor-beta are essential for the differentiation of interleukin 17-producing human T helper cells. Nature immunology 8(9):942-949; Bradshaw E M, et al. (2009) Monocytes from patients with type 1 diabetes spontaneously secrete proinflammatory cytokines inducing Th 17 cells. J Immunol 183(7):4432-4439). Significantly increased expression of both IL-1β and IL-6 was found in the supernatants of co-cultures containing both monocytes and T cells, but not TNF-α (FIG. 6A), IFN-γ, or IL-23 (data not shown). Both IL-1β and IL-6 were produced by monocytes (FIG. 6B). IL-1β and IL-6 were neutralized with neutralizing antibodies and it was found that the induction of IL-22 and IL-17 by C5a were significantly dampened (FIG. 2D). Collectively, these results indicate that not only direct interaction between monocytes and T cells, but also the secretion of IL-1β and IL-6 by monocytes is required for promotion of Th17 cytokines by C5a.

It has been well recognized that STAT3, RORC, and RORA are transcriptional regulators driving Th17 differentiation; however, the microarrray data did not suggest any changes in mRNA expression for these three factors. In contrast, BATF, which has been shown to promote Th17 cell differentiation (Bradshaw E M, et al. (2009) Monocytes from patients with type 1 diabetes spontaneously secrete proinflammatory cytokines inducing Th17 cells. J Immunol 183(7):4432-4439; Schraml B U, et al. (2009) The AP-1 transcription factor Batf controls T(H)17 differentiation. Nature 460(7253):405-409), was selectively induced by C5a in cells from AMD patients (FIG. 1A). As shown in FIG. 1B/D, C5a induced IL-22/IL-17 expression from T cells in both AMD patients and controls. To detect whether IL-1β and IL-6 are important for BATF expression, CD4⁺ T cells were stimulated from control donor with IL-1β and IL-6 in vitro. As shown in FIG. 2E, BATF expression was increased in the presence of IL-1β and IL-6, suggesting that the induction of IL-22 and IL-17 in CD4⁺ T cells could be at least partially due to the induction of BATF by monocyte derived IL-1β and IL-6.

C5a Protects T Cells from Undergoing Apoptosis.

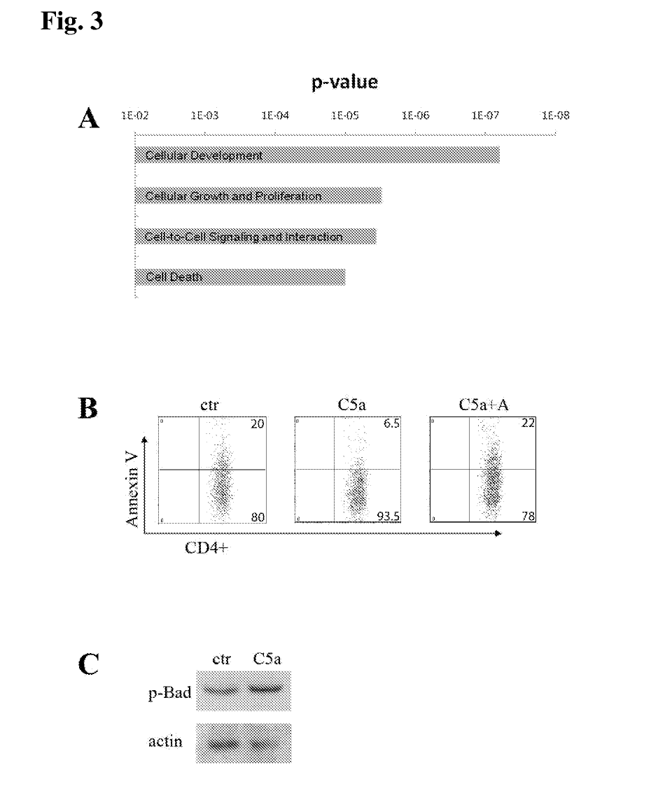

To fully understand the overall effect of C5a on CD4⁺ T cells from AMD patients, gene ontology enrichment analysis was performed using the EASE program on those 132 genes whose expression were differentially induced by C5a between healthy controls and AMD patients. Intriguingly, cell growth and proliferation, as well as cell death, are among the top 4 gene ontology categories that are significantly enriched within the list of 132 genes (FIG. 3A). C5a's effect on CD4⁺ T cell survival was examined. Purified PBMC cells naturally undergo apoptosis in culture and they usually die without stimulation in 7 days. C5a, with or without the C5aR, antagonist was added to the culture for 2 days and the percentage of cells undergoing apoptosis for more than 10 AMD patients, as well as controls, was compared. FIG. 3B represents a typical flow cytometry apoptosis staining. The addition of C5a prevented CD4⁺ T cells from undergoing apoptosis, as indicated by annexin V staining. This effect was abrogated by the addition of a C5aR antagonist. Moreover, the expression of phospho-Bad, one of the anti-apoptotic indicators, was increased in CD4⁺ T cells after C5a treatment (FIG. 3C).

Higher IL-22 and IL-17 Expression in the Serum of AMD Patients.

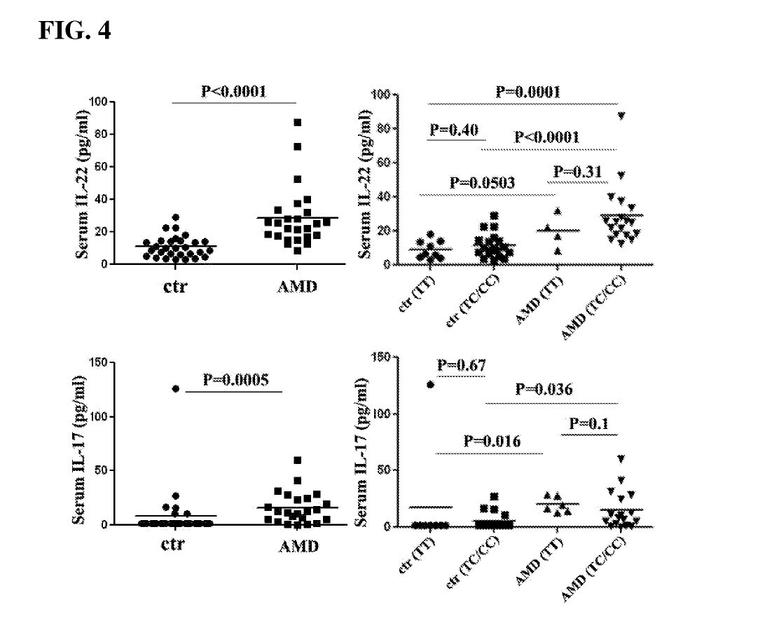

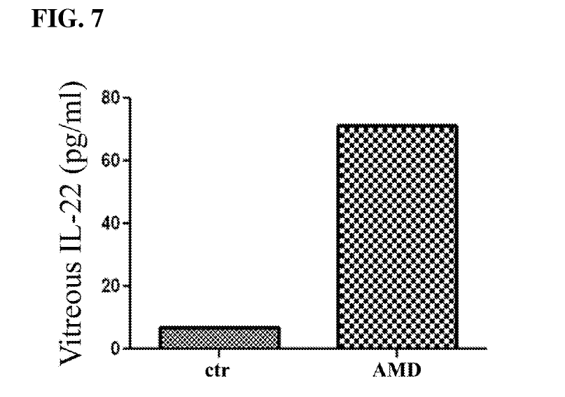

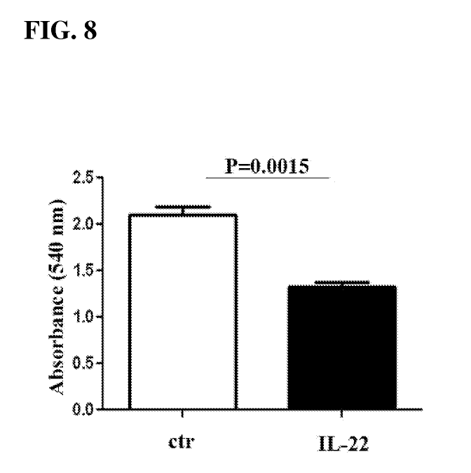

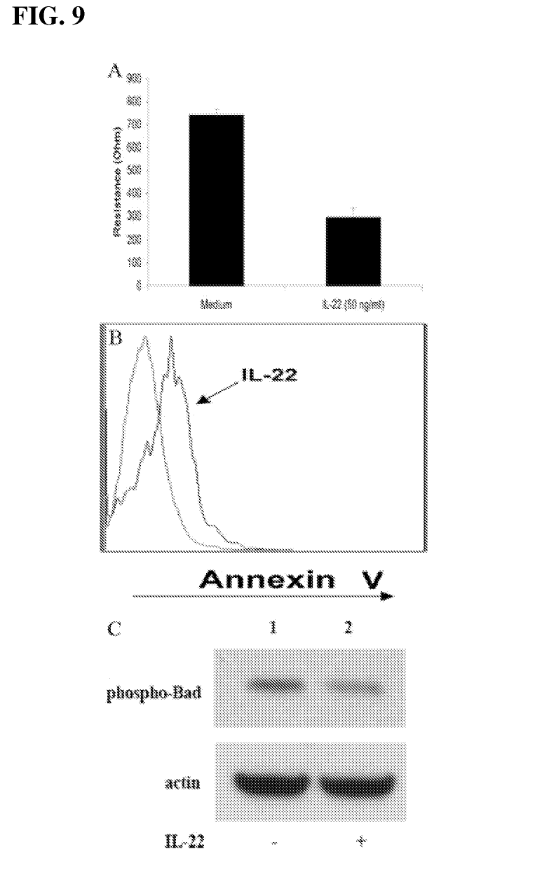

Elevated C5a levels have been reported in the serum of AMD patients (Scholl HP, et al. (2008) Systemic complement activation in age-related macular degeneration. PloS one 3(7):e2593). IL-22 and IL-17 levels in the serum of AMD patients was evaluated and as shown in FIG. 4, IL-22 and IL-17 levels were significantly elevated in AMD patients compared with controls. Cytokine expression in both the controls and the AMD patients was subgrouped based on their CFH SNP information (rs1061170). As shown in FIG. 4, cytokine high expression AMD patients have the risk CFH allele genotypes (heterozygous/homozygous, TC/CC). However, for control group, IL-22/IL-17 expressions were kept low regardless of their CFH SNP genotypes. elevated IL-22 expression was observed in one AMD patient's vitreous (FIG. 7) who had undergone ocular surgery for a macular hole. However, IL-17 was not detected in the vitreous of this patient (data not shown). Furthermore, it was found that IL-22 can affect primary pigment epithelial cells by decreasing their total tissue resistance and inducing apoptosis (FIGS. 8 and 9).

### REFERENCES

1. Ferris F L, 3rd, Fine S L, & Hyman L (1984) Age-related macular degeneration and blindness due to neovascular maculopathy. Archives of ophthalmology 102(11):1640-1642.
2. Nussenblatt R B, Liu B, & Li Z (2009) Age-related macular degeneration: an immunologically driven disease. Curr Opin Investig Drugs 10(5):434-442.
3. Patel M & Chan C C (2008) Immunopathological aspects of age-related macular degeneration. Seminars in immunopathology 30(2):97-110.
4. Edwards A O, et al. (2005) Complement factor H polymorphism and age-related macular degeneration. Science (New York, N.Y. 308(5720):421-424.
5. Hageman G S, et al. (2005) A common haplotype in the complement regulatory gene factor H (HFI/CFH) predisposes individuals to age-related macular degeneration. Proceedings of the National Academy of Sciences of the United States of America 102(20):7227-7232.
6. Klein R J, et al. (2005) Complement factor H polymorphism in age-related macular degeneration. Science (New York, N.Y. 308(5720):385-389.
7. Scholl H P, et al. (2008) Systemic complement activation in age-related macular degeneration. PloS one 3(7):e2593.
8. Nozaki M, et al. (2006) Drusen complement components C3a and C5a promote choroidal neovascularization. Proceedings of the National Academy of Sciences of the United States of America 103 (7):2328-2333.
9. Lalli P N, et al. (2008) Locally produced C5a binds to T cell-expressed C5aR to enhance effector T-cell expansion by limiting antigen-induced apoptosis. Blood 112(5):1759-1766.
10. Strainic M G, et al. (2008) Locally produced complement fragments C5a and C3a provide both costimulatory and survival signals to naive CD4+ T cells. Immunity 28(3):425-435.
11. Caspi R (2008) Autoimmunity in the immune privileged eye: pathogenic and regulatory T cells. Immunol Res 42(1-3):41-50.
12. Weaver C T, Hatton R D, Mangan P R, & Harrington L E (2007) IL-17 family cytokines and the expanding diversity of effector T cell lineages. Annu Rev Immunol 25:821-852.
13. Zheng Y, et al. (2007) Interleukin-22, a T(H)17 cytokine, mediates IL-23-induced dermal inflammation and acanthosis. Nature 445(7128):648-651.
14. Manel N, Unutmaz D, & Littman D R (2008) The differentiation of human T(H)-17 cells requires transforming growth factor-beta and induction of the nuclear receptor RORgammat. Nature immunology 9(6):641-649.
15. Volpe E, et al. (2008) A critical function for transforming growth factor-beta, interleukin 23 and proinflammatory cytokines in driving and modulating human T(H)-17 responses. Nature immunology 9(6):650-657.
16. Wilson N J, et al. (2007) Development, cytokine profile and function of human interleukin 17-producing helper T cells. Nature immunology 8(9):950-957.
17. Yang L, et al. (2008) IL-21 and TGF-beta are required for differentiation of human T(H)17 cells. Nature 454(7202):350-352.
18. Acosta-Rodriguez E V, Napolitani G, Lanzavecchia A, & Sallusto F (2007) Interleukins 1beta and 6 but not transforming growth factor-beta are essential for the differentiation of interleukin 17-producing human T helper cells. Nature immunology 8(9):942-949.
19. Bradshaw E M, et al. (2009) Monocytes from patients with type 1 diabetes spontaneously secrete proinflammatory cytokines inducing Th17 cells. J Immunol 183(7):4432-4439.
20. Martinez G J & Dong C (2009) BATF: bringing (in) another Th17-regulating factor. Journal of molecular cell biology 1(2):66-68.
21. Schraml B U, et al. (2009) The AP-1 transcription factor Batf controls T(H)17 differentiation. Nature 460(7253):405-409.
22. Yanamadala V & Friedlander R M (Complement in neuroprotection and neurodegeneration. Trends in molecular medicine 16(2):69-76.
23. Li Z, et al. (2008) Gene expression profiling in autoimmune noninfectious uveitis disease. J Immunol 181(7):5147-5157.
24. Fang C, Zhang X, Miwa T, & Song W C (2009) Complement promotes the development of inflammatory T-helper 17 cells through synergistic interaction with Toll-like receptor signaling and interleukin-6 production. Blood 114(5):1005-1015.
25. Liu J, et al. (2008) IFN-gamma and IL-17 production in experimental autoimmune encephalomyelitis depends on local APC-T cell complement production. J Immunol 180(9):5882-5889.
26. Xu R, et al. (2010) Complement C5a regulates IL-17 by affecting the crosstalk between DC and gammadelta T cells in CLP-induced sepsis. Eur J Immunol 40(4):1079-1088.
27. Hueber A J, et al. (2010) Mast cells express IL-17A in rheumatoid arthritis synovium. J Immunol 184(7):3336-3340.
28. Hubschman J P, Reddy S, & Schwartz S D (2009) Age-related macular degeneration: current treatments. Clinical ophthalmology (Auckland, N.Z 3:155-166.
29. Hu M. et al. (2011) C5a contributes to intraocular inflammation by affecting retinal pigment epithelial cells and immune cells. Br. J. Ophthalmol. December; 95(12)1738-44.
30. Liu B. et al. (2011) Complement component C5a promotes expression of IL-22 and IL-17 from human T cells and its implication in age-related macular degeneration. J. Translational Med. 9:111, 1-12.

What is claimed:

1. A method of treating age-related macular degeneration in a patient comprising administering to the patient a therapeutically effective amount of an anti-IL-22 antagonist, an anti-IL-17 antagonist, or a combination thereof.

2. The method of claim 1, wherein the age-related macular degeneration is wet age-related macular degeneration.

3. The method of claim 1, wherein the age-related macular degeneration is dry age-related macular degeneration.

4. The method of claim 1, wherein the anti-IL-22 antagonist is an anti-IL-22 antibody.

5. The method of claim 1, wherein the anti-IL-22 antagonist is a small molecule.

6. The method of claim 1, wherein the anti-IL-17 antagonist is an anti-IL-17 antibody.

7. The method of claim 1, wherein the anti-IL-17 antagonist is a small molecule.

8. An assay for use in a patient having or suspected of having macular degeneration, comprising:
determining the concentration of IL-22 in a serum sample from said patient, wherein an elevation in the level of IL-22 in said serum sample, relative to the concentration of IL-22 in the serum of patients not having macular degeneration, is indicative of the presence or severity of age-related macular degeneration in said patient.

9. An assay for use in a patient having or suspected of having macular degeneration, comprising:
determining the concentration of IL-17 in a serum sample from said patient, wherein an elevation in the level of IL-17 in said serum sample, relative to the concentration of IL-17 in the serum of patients not having macular degeneration, is indicative of the presence or severity of age-related macular degeneration in said patient.

10. A method of diagnosing the presence or severity of age-related macular degeneration comprising
determining the concentration of IL-22 and/or IL-17 in a serum sample from a patient having or suspected of having macular degeneration, wherein an elevation in the level of IL-22 and/or IL-17 in said serum sample, relative to the concentration of IL-22 and/or IL-17 in a control from an individual or individuals not having macular degeneration, is indicative of the presence or severity of age-related macular degeneration in said patient.

11. A method of diagnosing the presence or severity of age-related macular degeneration comprising
determining the concentration of IL-22 and/or IL-17 in a serum sample from a patient having or suspected of having macular degeneration,
determining whether the serum sample has an elevation in the level of IL-22 and/or IL-17, relative to the concentration of IL-22 and/or IL-17 in a control from an individual or individuals not having macular degeneration, the elevation being indicative of the presence or severity of age-related macular degeneration in said patient.

12. Use of an anti-IL-22 antagonist, an anti-IL-17 antagonist, or a combination thereof for treating age-related macular degeneration.

13. The use according to claim 12, wherein the age-related macular degeneration is wet age-related macular degeneration.

14. The use according to claim 12, wherein the age-related macular degeneration is dry age-related macular degeneration.

15. The use according to claim 12, wherein the anti-IL-22 antagonist is an anti-IL-22 antibody.

16. The use according to claim 12, wherein the anti-IL-22 antagonist is a small molecule.

17. The use according to claim 12 wherein the anti-IL-17 antagonist is an anti-IL-17 antibody.

18. The use according to claim 12, wherein the anti-IL-17 antagonist is a small molecule.
